(19) United States
(12) Patent Application Publication
(10) Pub. No.: US 2015/0216448 A1
(43) Pub. Date: Aug. 6, 2015

(54) SYSTEM AND METHOD FOR MEASURING LUNG CAPACITY AND STAMINA

(71) Applicant: COUNTINGAPP MEDICAL LTD., Tel Aviv (IL)

(72) Inventors: Chaim Lotan, Jerusalem (IL); Aviv Lotan, Tel Aviv (IL); Sigal Kremer-Tal, Ramat HaSharon (IL)

(21) Appl. No.: 14/426,301

(22) PCT Filed: Aug. 26, 2013
(86) PCT No.: PCT/IB2013/056883

§ 371 Date: Mar. 5, 2015

Related U.S. Application Data
(60) Provisional application No. 61/696,824, filed on Sep. 5, 2012.

(51) Int. Cl. A61B 5/087; A61B 7/00; A61B 5/00

(52) U.S. Cl. CPC A61B 5/087; A61B 5/7278; A61B 5/742; A61B 5/746; A61B 7/00; A61B 5/6898

(57) ABSTRACT

A computerized method and system for measuring a user's lung capacity and stamina, to detect Chronic Heart Failure, COPD or Asthma, comprising: providing a client application on a user's mobile communication device, said client application comprising executable computer code for: instructing the user to fill his lungs with air and utter vocal sounds within a certain range of loudness (decibels) while exhaling; receiving and registering by the mobile communication device said user's vocal sounds; stopping the registering of vocal sounds; measuring the length of the vocal sounds receiving time within said range of loudness, said time proportionate the user's lung volumes; and displaying the length of sound received time results on the mobile communication device screen.

### CROSS-REFERENCE TO RELATED PATENT APPLICATIONS

This patent application claims priority from and is related to U.S. Provisional Patent Application Ser. No. 61/696,824, filed Sep. 5, 2012, this U.S. Provisional Patent Application incorporated by reference in its entirety herein.

### TECHNICAL FIELD

The disclosure generally relates to remote medical diagnostic and monitoring systems and solutions and more specifically, to a system and method for performing remote monitoring for chronic heart failure patients, using a lung capacity and stamina examination conducted only with a mobile communication device application.

### BACKGROUND

Telemedicine systems have been proposed as a way of remotely diagnosing and treating patients using telephonic communications. However, known systems typically require the use of a specific medical device, such as wearable ECG monitors, web-based stethoscopes, web-connected cuffs monitor hypertension and the like.

In the fast evolvement of mobile communication devices such as Smartphones and tablet computers, these devices take on ever growing functionality as new applications are developed. Smartphones already serve as personal digital assistants (PDA) media players, digital cameras, video cameras, GPS navigation units, credit cards, multimedia hubs, gaming device and much more.

It would be desirable to make use of the various sensors built into Smartphones to enable at least some of the medical diagnosis functionality without the need for additional equipment.

In the field of diagnosing lung condition for example, several products have been offered for remote (or home) diagnosis, such as Spiro PD, available from PMD Healthcare (www.spiropd.com), Microlife PF 100, available from Microlife (http://www.microlifeusa.com) and others.

These devices are actual spirometers electrically connectable to a computer running a dedicated application that receives the spirometer readings and performs various analyses thereon, generate alerts, and/or transmits them electronically to the physician.

Oxford University researchers have developed a portable spirometer and phone application (http://www.isis-innovation.com/licensing/7358.html). This technology enables remote monitoring, assessment and diagnosis of asthma and chronic obstructive pulmonary disease (COPD). Here again an actual spirometer is connected with a telephone, to capture and transmit the spirometer readings.

In the field of diagnosing heart condition, some Apps are available such as AliveCor (http://www.alivecor.com/) or SmartHeart (http://www.shl-telemedicine.com/portfolio/smartheart/) for ECG measurements, also using sensors that are attached to the smartphone.

The association between lung volumes and chronic heart failure has been commonly acceptable [Ref—Congestive Heart Failure: Diagnosis, Pathophysiology, Therapy, and Implications for Respiratory Care, respiratory care, April 2006 Vol 51 no 4, pp. 403-412—incorporated by reference in its entirety herein], as deterioration in heart function leads to fluid retention, mainly in the lungs. As a result, there is less available space for air in the alveoli, as it is taken up by fluid. The degree of this process is linked to the severity of heart failure.

As a result of the fluid retention, one of the first symptoms presented by the patient is shortness of breath. For that matter, the invention uses a simple breath test to monitor and identify such deterioration in heart function.

The breath test, based on a speech/vocal measurements by the smartphone's microphone, provides an accurate and sensitive measurement to alert patient or caregiver when early signs of deterioration are tracked.

### SUMMARY

According to a first aspect of the present invention there is provided a computerized service implementing a method of measuring a user's lung capacity and stamina, to detect Chronic Heart Failure, COPD or Asthma, comprising: providing a client application on a user's mobile communication device, said client application comprising executable computer code for: instructing the user to fill his lungs with air and utter vocal sounds within a certain range of loudness (decibels) while exhaling; receiving and recording by the mobile communication device said user's vocal sounds; stopping the recording of vocal sounds; measuring the length of the vocal sounds receiving time within said range of loudness, said time proportionate the user's lung volumes; and displaying the length of sound received time results on the mobile communication device screen.

The range may be determined by a personal calibration.

Stopping the recording of vocal sounds may be initiated by the user.

Stopping the recording of vocal sounds may be automated by the application when no voice has been received during a pre-defined time.

The method may additionally comprise, during the step of receiving vocal sounds, displaying on the mobile communication device screen a graphic representation of the measurements.

The method may additionally comprise sharing the measurements with other persons using the Internet.

Sharing may comprise sending by email.

Sharing may comprise publishing to a social network.

The method may additionally comprise registering the user to the service and storing the registered user's measured results in a database residing on a sever, said server running a server application communicating with said client application.

Registering may comprise defining a daily reminder for carrying out the measurements.

Registering may comprise providing at least one contact information other than the user's.

The method may additionally comprise providing rewards to loyal users.

The method may additionally comprise providing by said server application a history of the user's measurements, communicating said history to said client application and displaying said history by said client application on the mobile communication device screen.

Displaying the history may comprise displaying statistics relevant to various clinical conditions.

The clinical conditions may comprise at least one of rest, following physical activity, medication change and hospitalization.

The method may additionally comprise computing by said server application statistics related to lung capacity and stamina over large populations using said database.

According to a second aspect of the present invention there is provided a system for measuring a user's lung capacity and stamina to monitor chronic diseases, such as COPD, Asthma or Chronic Heart Failure, comprising: a server connected to a network, said server comprising a processor and configured to execute programmable commands of a server application and at least one database; at least one electronic mobile communication device communicating with said server over a network, said electronic communication device comprising a processor and configured to execute programmable commands of a user application; said electronic communication device additionally comprising a microphone and a display configured to provide GUI (Graphical User Interface) means for receiving user input and providing output to the user; said microphone configured to register said user's voice; said user GUI input means configured to: prompt the user to start a test; and display the test results; said user application configured to: display said GUI; start and stop said test; receive the user's voice during said measurements; and measure the time between said start and stop; said server application configured to: store in said at least one database measurements results of a plurality of users; communicate said results upon request to said user application; and compute statistics using said plurality of user measurements.

Receiving the user's voice may comprise recording said user's voice.

The GUI means may additionally be configured to display on said mobile communication device screen a graphic representation of the test.

The graphic representation may comprise a range of loudness.

The GUI means may additionally be configured to register a user to the system.

Registering may comprise defining a time for a daily reminder to perform the measurements.

Registering may comprise providing contact information of at least one person other than the user.

The GUI means may additionally be configured to display measurements results in various periods.

Stopping the test may be done automatically by the user application after a pre-defined time in which no voice has been received.

The system may additionally comprise a website, said website providing the user at least one of: viewing all his test results, performing his own statistical deductions and downloading the user application and redeeming loyalty credit.

The website may additionally comprise at least one of: relevant articles, links to online forums and/or other social networks and advertisement space.

According to a third aspect of the present invention there is provided a method of measuring a user's lung capacity and stamina, to detect Chronic Heart Failure, COPD or Asthma, comprising: instructing the user to fill his lungs with air and utter vocal sounds within a certain range of loudness (decibels) while exhaling; registering said user's vocal sounds; stopping the registering of vocal sounds; and measuring the length of the vocal sounds receiving time within said range of loudness, said time proportionate to the user's lung volumes and an indicator for the presence of fluid in the lungs.

According to a fourth aspect of the present invention there is provided a computer storage medium tangibly embodying a program of machine-readable instructions executable by a digital processing apparatus to perform the method of: providing a client application on a user's mobile communication device, said client application comprising executable computer code for: instructing the user to fill his lungs with air and utter vocal sounds within a certain range of loudness (decibels) while exhaling; receiving and recording by the mobile communication device said user's vocal sounds; stopping the recording of vocal sounds; measuring the length of the vocal sounds receiving time within said range of loudness, said time proportionate the user's lung volumes; and displaying the length of sound received time results on the mobile communication device screen.

### BRIEF DESCRIPTION OF THE DRAWINGS

In order to understand the invention and to see how it may be implemented in practice, a plurality of embodiments will now be described, by way of non-limiting example only, with reference to the accompanying drawings, in which:

FIG. 1 is a schematic representation of an exemplary system for carrying out the present invention;

FIG. 2 is a flowchart showing the main steps performed by the method of the present invention; and

FIGS. 3A through 3H show an exemplary Graphical User Interface (GUI) of the client application, for performing the method of FIG. 2.

### DETAILED DESCRIPTION OF PREFERRED EMBODIMENTS

The present invention provides an easy to use system and method for performing a remote medical lung capacity and stamina examination using measurements of the time length a patient is capable of uttering a sound without inhaling, preferably incorporated in a mobile communication device application, without the use of any additional device, medical or other.

Potential users of the present invention are already diagnosed patients with a chronic medical condition such as Heart Failure, Chronic Obstructive Pulmonary Disease (COPD), including Asthma who may use the system frequently and in any location, without the need to visit the physician.

Other potential users may be athletes, or generally, people doing physical training, who wish to measure the increase in their lung stamina, or people who wish to test their lung stamina or capacity for any other reason.

Test is performed by guiding the user to utter voice at a constant loudness through principles of incentive spirometry. This results in exhalation at a constant flow. The length of such exhalation is equivalent to the patient's expiratory volume. The user is guided to inhale to his/her full capacity before said exhalation, up to complete emptying of the lungs. This volume is equivalent to Forced Vital Capacity (FVC).

The first use may, for example, take place when the user is discharged from hospital (in relatively good/stable condition). From then on the application monitors changes and generates early enough alerts, in order to prevent subsequent hospitalizations.

Lung volume signature is usually personal and ranges from patient to patient. It is dependent on age, height, BMI, lung diseases, thorax anatomic variations and genetics. So, as best use for the application, it is recommended that the patient performs regular measurements, thus generating specific signature of own-lung volume stored in the system. The user's “lung volume” is defined as his personal “Baseline”, that will reflect his performance in stable condition. Baseline is a fixed parameter for each patient, although over time it may be subjected to gradual drift upon changes in weight, heart and lung condition, etc (in which case the patient will have to determine a new baseline). Any significant deviation from current baseline will be registered and subsequently alerted. A detection of a decrease in patient typical baseline results, analyzed by the application server, may initiate an alert to at least one of the following:
a. The patient.
b. The caregiver (relative and/or professional).
c. The HMO and/or other remote monitoring service provider.

d. Personal physician.

e. Insurance company.

f. Nearest hospital and/or attending hospital.

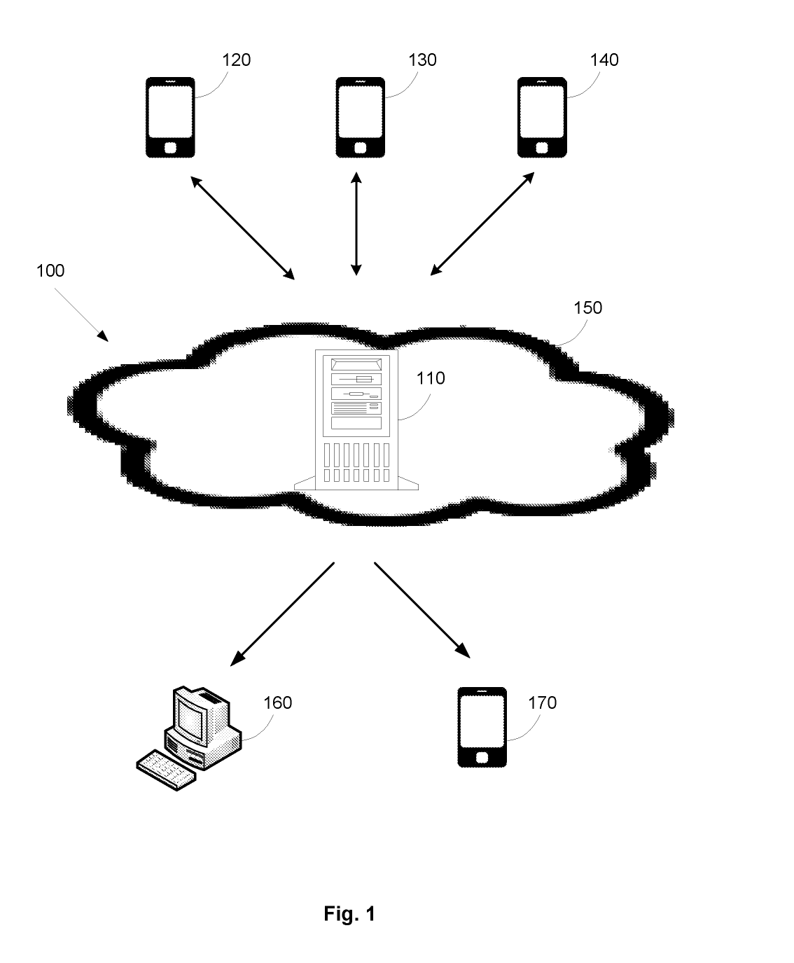

FIG. 1 is a schematic representation of an exemplary system for carrying out the present invention.

The system 100 comprises a system server 110, communicating bi-directionally with a plurality of user (patient) mobile communication devices, e.g. smartphones (120, 130, 140) over a communications network 150, such as the Internet. The system server 110 also communicates with selected overseeing entities such as a physician, a care taker, a family member, etc. using personal computers 160 or mobile communication devices 170, over a communications network 150, such as the Internet.

The user mobile communication devices (120, 130, 140) run a client application designed to measure lung capacity and stamina, as will be explained in detail below. The application may be downloaded from the server 110 or from any application downloading server such as Appstore (iOS platform) or Market (for Android platform) and the like.

The server 110 comprises a server application designed to communicate with the user application, as will be explained in detail below. The server 110 additionally comprises one or more databases for storing users' test data. The server 110 may additionally comprise an application website.

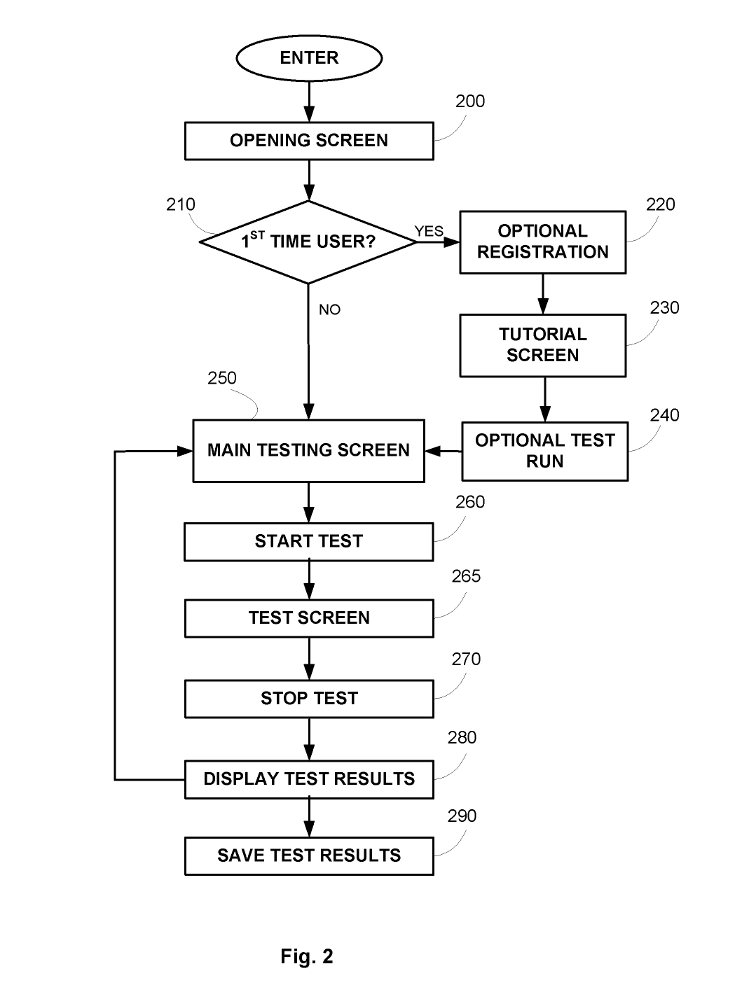

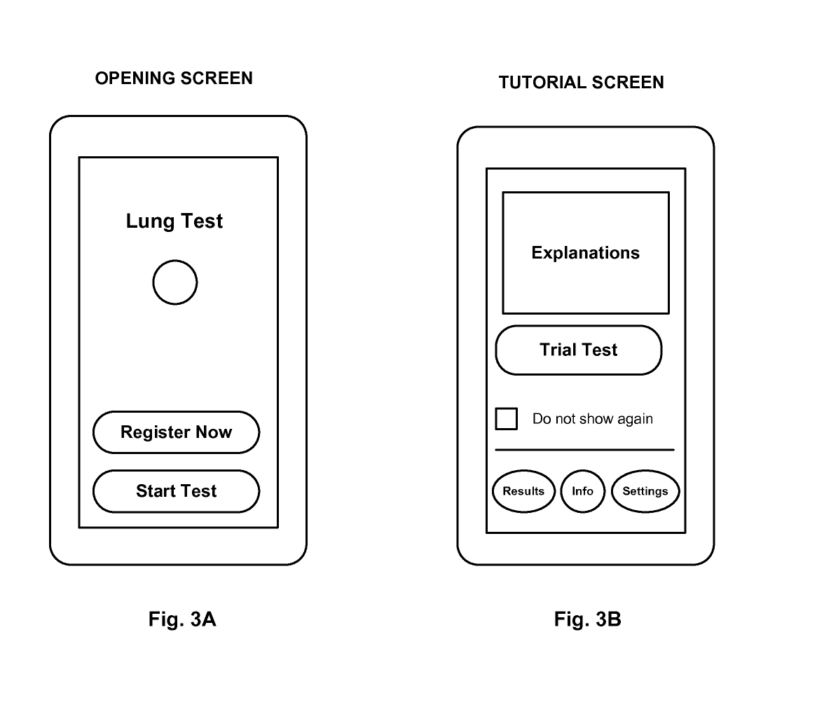

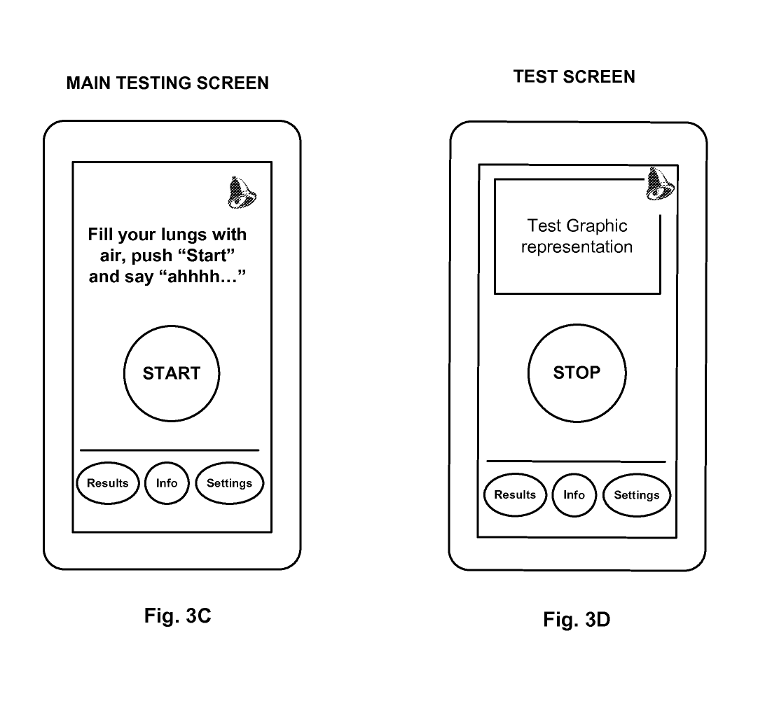

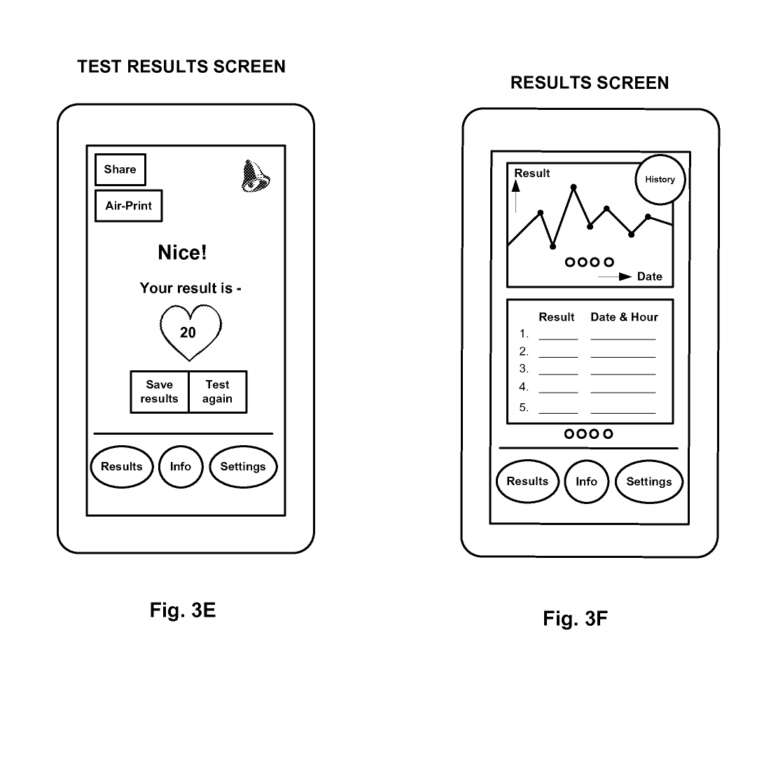

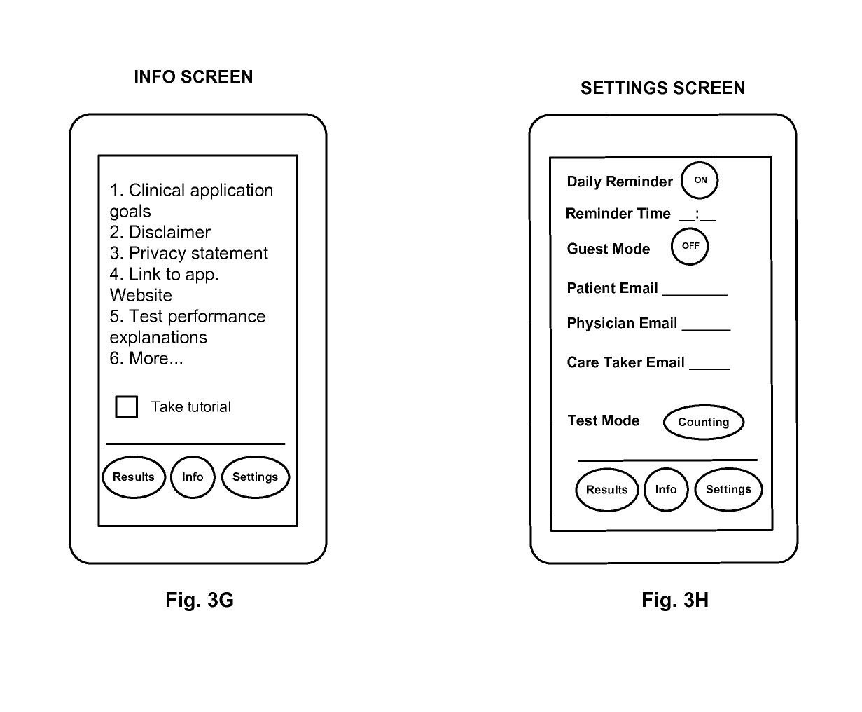

Attention is drawn now to FIG. 2, in conjunction with FIGS. 3A through 3H.

FIG. 2 is a flowchart showing the main steps performed by the method of the present invention.

FIGS. 3A through 3H show an exemplary Graphical User Interface (GUI) of the client application, for performing the method of FIG. 2.

In step 200 the client application's opening screen is displayed, as shown in FIG. 3A. The user may be a new user wishing to register to the system, a returning user already registered and familiar with the application, or a “guest” wishing to sample the application without registration.

As shown in FIG. 3A, a new user wishing to register to the system may select the “Register Now” button (step 220), resulting in the display of a registration form, in which the user may choose to receive a daily reminder to perform the test at a pre-defined hour. The user is prompted to provide contact information (e.g. e-mail address or telephone number) for himself, his physician and a care giver or family member. Failure to perform the test at or near the designated daily hour may cause a notification (e.g. by email) to be automatically sent by the system to at least one of the provided contacts.

The user may also be prompted to provide a voice signature, which may be analyzed by voice analysis software during the test performance, to determine:
a. Authentication for privacy.
b. Authentication for receiving only the user's/patient's voice and avoiding receiving and saving guest's tests.
c. More accurate test results.

The application may sample the user's voice during a first few number of test runs (2-3 tests), to determine a personal, customized range of decibels for that user, thus improving the personalization of user's baseline test results.

The user's mobile communication device number may be provided by the user or automatically identified and saved for future identification purposes.

At the end of the registration process, the new user's data is uploaded to the system server 110 and the application displays the tutorial screen (FIG. 3B, step 230).

If the user does not wish to register to the system (e.g. guest), he may select the “Start Test” button, upon which the tutorial screen (FIG. 3B) is displayed (step 230) or, if the user is a returning user, the main testing screen (FIG. 3C) is displayed (step 250).

The exemplary tutorial screen shown in FIG. 3B may comprise explanations as to the nature and goals of the test and an option to initiate a trial test. The user may check the “Do not show again” box if he does not wish for the tutorial screen to be displayed in the future.

The main testing screen (FIG. 3C, step 250) comprises specific directives for starting the test. In the example of FIG. 3C, the user is directed to fill his lungs with air, push the “Start” button and say “ahhhh” (=exhalation of the full volume of his lung) as long as he can, without inhaling. In an alternative embodiment of the testing method, the user may be directed to start counting from 1 upward as long as he can without inhaling, instead of saying “ahhhh”. The user may select the testing mode e.g. by indicating it on the settings screen. In the exemplary settings screen of FIG. 3H, the test mode selection is implemented as a toggle between the two modes.

Once the start button has been pushed (step 260), the application displays the test screen (FIG. 3D, step 265). The test screen may comprise a graphic representation of the test, such as a bar showing voice intensity, numbers to be voiced, length of time of exhalation, or any other graphic representation appropriate for providing the user with test feedback.

According to an embodiment of the invention, the graphic representation of the test may comprise upper and lower limiters depicting a range of loudness within which the user's voice has to remain while taking the test. This range may be personalized, as explained above. Deviation from the assigned range may result in the test being declared as “Failed” (or not-accurate) and the user may be prompted to run the test again.

The test is stopped (step 270) by the user pushing the “Stop” button. Alternatively, the test may be automatically stopped when the application detects e.g. 2 seconds of silence.

Following the test termination, a test result screen is displayed (FIG. 3E, step 280). The test result screen may comprise a numerical result (e.g. number of seconds) accompanied by an evaluation of the result (e.g. Nice). If the result is poor (e.g. significantly lower than previous results stored for the user), the user may be prompted to run the test again.

At first use, the user is asked to perform the test under predetermined conditions (at rest, in the morning, in quiet surroundings etc.) e.g. three times, in order to determine a personalized standard. Hence, the application needs to determine a personal “Baseline”, as a starting point. This reflects the patients “normal” performance to be compared with future results which may change due to deterioration in patient's condition.

After “Baseline” has been set, under predetermined conditions, the user will conduct all future tastings under the same conditions. If a certain test will show a ‘suspicious result’, as will be explained below, the patient will have to address a questionnaire that will determine whether the deviation may have occurred due to a test taken under different conditions than those instructed for determining the “Baseline” (e.g. after physical activity, change of medication etc.).

The length of time in which the user has uttered vocal sounds within a predetermined loudness range is proportionate to the volume of air flow from the lungs and may thus be used as a volume measuring device (e.g. incentive spirometer), without the need for an actual device other than the mobile communication device:

### Constant Air Flow×Time=Volume

As the patient is instructed to fill his lungs to the fullest, and then exhale to the fullest extent, at a constant flow (clamped to a certain loudness range), the test is equivalent to Forced Vital Capacity (FVC).

Automatic identification of an invalid test is generated. Invalid test results may be obtained when the user did not perform the test under the predetermined conditions; user intermittent inhalation during exhalation measurement; when user did not follow predetermined loudness range (e.g., variable distance from the mouth). Then a questionnaire is presented to the user, to identify immediate addressable causes for invalid test results and ways to correct them. Subsequently, an additional test may be suggested, either immediately, or later, or following rest, according to relevant circumstances.

If results inconsistent with the baseline test results are repeated, an alert is generated and care-provider attention is suggested.

After finishing a valid measurement several options may be suggested:
A “Share” button may enable the user to share his test results by email, Facebook, Twitter, or any network or sharing medium with a pre-selected group.
An “Air-print” button may enable the user to remotely print his test results to the back-end service or to his own printer.
Selecting the “Test again” button is followed by re-display of the main testing screen (FIG. 3C).
Selecting the “Save results” screen causes the results to be uploaded to the server (step 290) and the Results screen (FIG. 3F) to be displayed.

The exemplary result screen shown in FIG. 3F comprises a graphic display of the test results by date, where selecting a point on the graph may display the selected date and test result. The graph may additionally be manipulated by “pinching” or “expanding” it, to change the results' time span (e.g. from weekly to yearly). The graph may additionally comprise scroll capabilities to show different periods.

The result screen may further comprise a table showing numerical test results in e.g. last month. The results table may also be scrollable to show next or previous months' results.

A “History” button may be provided, which transfers the user to his personal zone in the application website for viewing all his test results.

“History” may also provide classification of tests according to user's state (e.g. after rest or after physical exertion or days after discharge from hospital, after a change of treatment or medication, etc.).

The screens of FIGS. 3B through 3F show three additional buttons: “Results” “info” and “Settings”.

Selecting the “Results” buttons from anywhere in the application will cause the results screen (FIG. 3F) to be displayed.

Selecting the “Info” button from anywhere in the application will cause the info screen (FIG. 3G) to be displays. The info screen may comprise various informative items relating to the testing application such as, for example, the clinical application goals, a disclaimer, a privacy statement, a link to the application website, explanations regarding the test performance and more. The info screen may additionally comprise a “Take tutorial” checkbox, the checking of which will be equivalent to un-checking the “Do not show again” checkbox in the tutorial screen (FIG. 3B). The info screen may additionally comprise a verbal explanation of how to take the test.

Selecting the “Settings” button from anywhere in the application will cause the setting screen (FIG. 3H) to be displays. The settings screen may serve for updating or editing information provided during the registration process, such as turning on/off the daily reminder feature, providing contact information for the patient, the physician and a care giver or family member and defining “Guest mode” for a test.

Test results of tests run under “Guest” mode are not saved.

The “Results”, “Info” and “Settings” buttons are inactive during the test performance.

A “Reminder” icon, shown as a bell in Figs, 3C through 3E, may serve for displaying the settings screen for establishing a daily test reminder.

The system's database comprising information and test results of a plurality of patients may be used by the system application to perform various statistical operations for calculating, for example, variance of a patient's test results between different times of testing, variance of test results between patients, variance of test results according to other known parameters (age, gender, known disease, geographic location, smoking and more).

The system's website may provide the registered user tools for performing their own statistical calculations.

The system's website may additionally comprise:
Relevant articles;
A link for downloading the user application;
Links to online forums and/or other social networks;
An online store enabling the use of credit earned by the loyalty program;
Advertisement space;
More

According to a further embodiment, the application may use the mobile communication device's camera to direct the user in accurately positioning the mobile device in front of him while performing the test, e.g. by displaying a frame in which the user is directed to see his face.

According to a further embodiment, the application may be infused with Game Mechanics (Gamification methods) in the form of a loyalty program, which is supposed to motivate the user to keep his/hers regimen of daily tests. The loyalty program may change each month/quarter and may feature scoreboards, leaderboards, teams, tokens, trophies, coupons, discounts of medical insurance fees and more.

According to a further embodiment, the application may be used as a game for children who are chronic patients and are potential users of the application. The game will include blowing balloons, but making sure they do not burst (thus keeping the user's voice at the suitable tone) and making yourself (kind of an avatar) walk a straight line or keep from falling off a ledge, etc.

1. A computerized service implementing a method of measuring a user's lung capacity and stamina, comprising:
providing a client application on a user's mobile communication device, said client application comprising executable computer code for:
a. instructing the user to fill his lungs with air and utter vocal sounds within a certain range of loudness (decibels) while exhaling;
b. receiving and registering by the mobile communication device said user's vocal sounds;
c. stopping the recording of vocal sounds; and
d. measuring the length of the vocal sounds receiving time within said range of loudness, said time proportionate the user's lung volumes.

2. The method of claim 1, wherein said range is determined by a personal calibration.

3. The method of claim 1, wherein said stopping the registering of vocal sounds is done automated by the application when no voice has been received during a pre-defined time.

4. (canceled)

5. The method of claim 1, additionally comprising, during said step of receiving vocal sounds, displaying on said mobile communication device screen a graphic representation of the measurements.

6-8. (canceled)

9. The method of claim 1, additionally comprising registering the user to the service and storing said registered user's measured results in a database residing on a server, said server running a server application communicating with said client application.

10. The method of claim 9, wherein said registering comprises one of defining a daily reminder for carrying out the measurements and providing at least one contact information other than the user's.

11. (canceled)

12. (canceled)

13. The method of claim 9, additionally comprising providing by said server application a history of the user's measurements, communicating said history to said client application and displaying said history by said client application on the mobile communication device screen.

14. The method of claim 13, wherein said displaying the history comprises displaying statistics relevant to various clinical conditions comprising at least one of rest, following physical activity, medication change and hospitalization.

15. (canceled)

16. The method of claim 9, additionally comprising computing by said server application statistics related to lung capacity and stamina over large populations using said database.

17. A system for measuring a user's lung capacity and stamina, comprising:
a server connected to a network, said server comprising a processor and configured to execute programmable commands of a server application and at least one database;
at least one electronic mobile communication device communicating with said server over a network, said electronic communication device comprising a processor configured to execute programmable commands of a user application;
said electronic communication device additionally comprising a microphone and means for receiving user input and providing output to the user;
said microphone configured to register said user's voice;
said user input means configured to:
prompt the user to start a test; and
display the test results;
said user application configured to:
provide feedback to the user;
start and stop said test;
receive and register the user's voice during said measurements; and
measure the time between said start and stop;
said server application configured to:
store in said at least one database measurements results of a plurality of users;
communicate said results upon request to said user application; and
compute statistics using said plurality of user measurements.

18-25. (canceled)

26. The system of claim 17, additionally comprising a website, said website providing the user at least one of: viewing all his test results, performing his own statistical deductions and downloading the user application and redeeming loyalty credit.

27. (canceled)

28. A method of measuring a user's lung capacity and stamina, comprising:
a. instructing the user to fill his lungs with air and utter vocal sounds within a certain range of loudness (decibels) while exhaling;
b. registering said user's vocal sounds;
c. stopping the registering of vocal sounds; and
d. measuring the length of the vocal sounds receiving time within said range of loudness, said time proportionate to the user's lung volumes and an indicator for the presence of fluid in the lungs.

29. A computer storage medium tangibly embodying a program of machine-readable instructions executable by a digital processing apparatus to perform the method of:
providing a client application on a user's mobile communication device, said client application comprising executable computer code for:
a. instructing the user to fill his lungs with air and utter vocal sounds within a certain range of loudness (decibels) while exhaling;
b. receiving and recording by the mobile communication device said user's vocal sounds;
c. stopping the recording of vocal sounds; and
d. measuring the length of the vocal sounds receiving time within said range of loudness, said time proportionate the user's lung volumes.

30. The method of claim 1, further comprising displaying the measured time length.

31. The method of claim 1, further comprising comparing the measured time length with a predefined baseline and issuing an alert accordingly.

32. The method of claim 1, wherein said measurement is used for monitoring one of Chronic Heart Failure, COPD and Asthma.

33. The method of claim 12, wherein said measurement is used for monitoring one of Chronic Heart Failure, COPD and Asthma.

34. The system of claim 10, wherein said measurement is used for monitoring one of Chronic Heart Failure, COPD and Asthma.
